(19) United States
(12) Patent Application Publication
(10) Pub. No.: US 2020/0103411 A1
(43) Pub. Date: Apr. 2, 2020

(54) Predicting Cancer Treatment Outcome With T-DM1

(71) Applicants: Expression Pathology, Inc., Rockville, MD (US); Memorial Sloan-Kettering Cancer Center, New York, NY (US)

(72) Inventors: Todd HEMBROUGH, Gaithersburg, MD (US); Fabiola CECCHI, Washington, DC (US); Sarit SCHWARTZ, Rockville, MD (US); Maurizio SCALTRITI, New York, NY (US); Bob T. LI, New York, NY (US)

(21) Appl. No.: 16/617,580

(22) PCT Filed: Jun. 4, 2018
(86) PCT No.: PCT/US2018/035836

§ 371 Date: Nov. 27, 2019

Related U.S. Application Data
(60) Provisional application No. 62/514,661, filed on Jun. 2, 2017.

(51) Int. Cl. G01N 33/574; A61K 47/68; A61K 31/5365; C12Q 1/6886

(52) U.S. Cl. CPC G01N 33/57492; A61K 47/6803; A61K 47/6849; C12Q 2600/106; C12Q 1/6886; G01N 2333/71; C12Q 2600/156; A61K 31/5365

(57) ABSTRACT

Improved methods for treating lung cancer are provided. Tumor samples from patients are analyzed (i) by DNA sequencing to detect the presence of HER2 mutations and (ii) by mass spectrometric proteomic analysis to determine whether HER2 protein is expressed in the tumor cells. Patients respond to therapy with trastuzumab emtansine (T-DM1) or an equivalent antibody-drug conjugate when unique HER2 protein fragments are detected in the patient's tumor cells that harbor HER2 mutations. Conversely, patients do not respond to T-DM1 therapy when the tumor cells contain HER2 mutations but the unique protein fragments are not detected. Detection of HER3 in the tumor cells is a positive predictor of response to treatment.

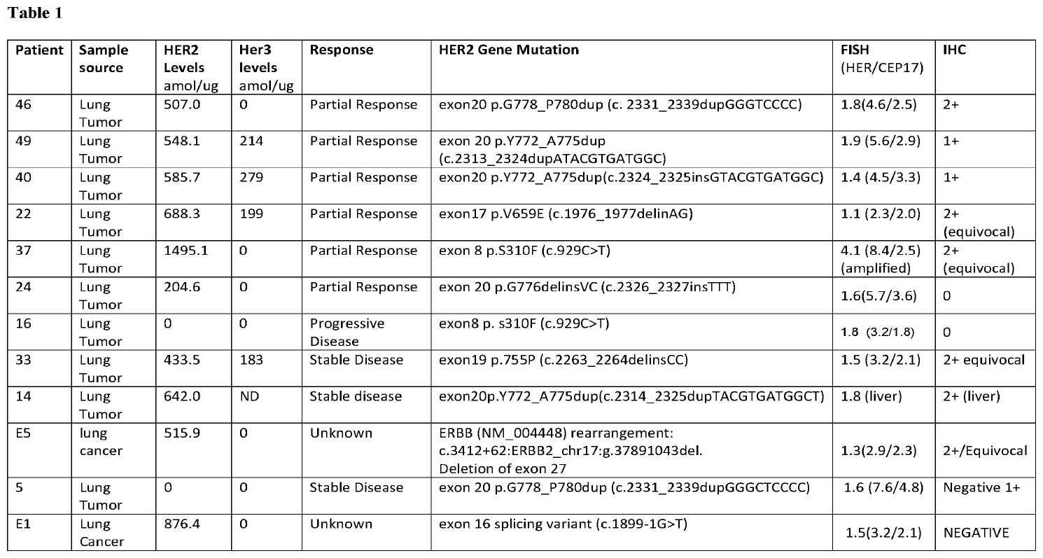

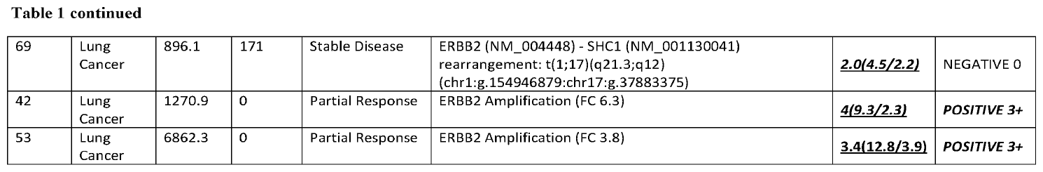

### INTRODUCTION

Methods are provided for treating cancer patients, and especially lung cancer patients, by assaying tumor tissue surgically-removed from patients and identifying those patients most likely to respond to treatment with HER2-targeted therapeutic agents, such as trastuzumab emtansine (T-DM1) and other anti-Her-2 antibody-drug conjugates. In tumors that are positive for at least one HER2 mutation, patients are treated with T-DM1 or an equivalent antibody-drug conjugate (“ADC”) when the tumors expresses a detectable amount of HER2 protein expression. This treatment is effective even when HER2 expression is below the level that is conventionally viewed as HER2-positive and/or where the HER2 gene is not amplified. Detectable expression of HER3 protein is also a predictor of the likelihood of response to T-DM1 or an equivalent ADC.

The HER2 protein, also known as human epidermal growth factor receptor 2, receptor tyrosine-protein kinase erbB-2, CD340 (cluster of differentiation 340), and proto-oncogene Neu, (hereinafter “HER2”), is a protein that in humans is encoded by the ERBB2 gene. HER2 is a member of the human epidermal growth factor receptor (HER/EGFR/ERBB) family. Amplification or over-expression of this oncogene has been shown to play an important role in the development and progression of certain aggressive types of breast cancer. In recent years the protein has become an important biomarker and target of therapy for approximately 30% of breast cancer patients.

The Her3 protein, also known as human epidermal growth factor receptor 3, and receptor tyrosine-protein kinase erbB-3, (hereinafter “Her3”), is a protein that in humans is encoded by the ERBB3 gene. Her3 is also a member of the human epidermal growth factor receptor (HER/EGFR/ERBB) family Like other ERBB family members, Her3 can homodimerize or heterodimerize with other members of the ERBB family. The HER2-Her3 heterodimer is the most active of the possible dimers, and the ligand-activated heterodimer activates multiple pathways including the MAPK, PI3K/Akt, and PLCγ pathways.

Trastuzumab emtansine, referred to here as T-DM1, and also known as ado-trastuzumab emtansine and T-DM1, is a therapeutic agent that specifically targets and binds to the HER2 protein. T-DM1 is sold under the trade name Kadcyla and is an antibody-drug conjugate where the monoclonal antibody trastuzumab (Herceptin) is linked to the cytotoxic agent emtansine (DM1). Trastuzumab alone stops growth of cancer cells by binding to the HER2 protein, allowing the conjugated DM1 to enter the cell and destroys them by binding to tubulin. Trastuzumab binding to HER2 prevents homodimerization or heterodimerization (HER2/Her3) of the receptor, ultimately inhibiting the activation of MAPK and PI3K/Akt cellular signaling pathways. Because the monoclonal antibody targets HER2, and HER2 is only over-expressed in cancer cells, the conjugate delivers the toxin preferentially to tumor cells. Other anti-HER2 antibody-drug conjugates are currently being developed and may also be used in the methods described herein. Reference herein to use of T-DM1 will be understood to include use of other anti-HER2 antibody-drug conjugates unless specifically indicated otherwise.

The method for identifying cancer patients, and in particular lung cancer patients, most likely to respond to treatment with trastuzumab emtansine (T-DM1), or other anti-HER2 antibody-drug conjugates, identifies patients whose tumors not only express the HER2 protein but also harbor a mutation in the HER2 gene. Tumor cells from a cancer patient are analyzed for HER2 expression and the presence of at least one HER2 mutation. When both are observed then the patient is treated with T-DM1. In addition, detection of HER3 expression in the tumor further indicates that the patient will benefit from treatment with T-DM1.

### SUMMARY OF THE INVENTION

What is provided is a method of treating a patient suffering from cancer, where the steps of the method include (a) detecting and quantifying the level of the HER2 protein in tumor cells obtained from the patient, where the tumor sample includes one or more mutations in the HER2 gene; (b) treating the patient with a first therapeutic regimen including an effective amount of the therapeutic agent trastuzumab emtansine (T-DM1) or an equivalent anti-HER2 antibody-drug conjugate when HER2 protein is detected, and where the HER2 protein is not overexpressed under the ASCO guidelines or (c) treating the patient with a second therapeutic regimen that does not include an effective amount of T-DM1 when HER2 is not detected. Optionally, the tumor cells may be tested for expression of HER3 protein, where the patient is treated with the first therapeutic regimen including an effective amount of the therapeutic agent trastuzumab emtansine (T-DM1) when HER2 expression is detected, and where HER2 is not overexpressed under the ASCO guidelines and when HER3 is detected. HER2 protein may be detected by detecting a HER2 fragment peptide by mass spectrometry in a protein digest of the tumor cells, and HER3 protein may be detected by detecting a HER3 fragment peptide by mass spectrometry in a protein digest of the tumor cells.

In these methods, the protein digest may include a protease digest, such as a trypsin digest. The mass spectrometry may include tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry, and the mode of mass spectrometry used can be Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

The tumor cells may be from solid tissue, which may be formalin fixed solid tissue and may also be paraffin embedded. Detecting a unique fragment peptide, or multiple unique fragment peptides, from the HER2 protein includes detecting and/or determining quantitative levels of unique fragment HER2 peptide, or multiple unique HER2 fragment peptides, in the sample by comparing to a spiked internal standard peptide of known amount, where both the native peptide in the biological sample and the internal standard peptide corresponds to the same amino acid sequence of the unique fragment peptide, or multiple unique fragment peptides, from the HER2 protein. The internal standard peptide, or multiple internal standard peptides, may be isotopically labeled peptide. And may include one or more heavy stable isotopes selected from ¹⁸O, ¹⁷O, ¹⁵N, ¹³C, ²H or combinations thereof.

These methods of detecting and quantitating a unique fragment peptide, or multiple unique fragment peptides, from the HER2 protein can be combined with detecting and quantitating other peptides from other proteins in multiplex so that the treatment decision about which agent used for treatment is based upon detection and/or quantitating a unique fragment peptide, or multiple unique fragment peptides, from the HER2 protein in combination with other peptides/proteins in the biological sample.

The mutation, or mutations, may be detected within the HER2 gene in a biological sample prepared from tumor cells obtained from the patient using one or more methods such as standard nucleic acid sequencing, next generation nucleic acid sequencing, polymerase chain reaction, restriction fragment polymorphism analysis, fluorescent in-situ hybridization (FISH), and combinations of these. The DNA mutation in the HER2 gene within the tumor cells may be, for example one or more of single nucleotide changes, insertions, deletions, rearrangements, duplications, duplications/deletions of individual nucleotides, duplications/deletions of multiple nucleotides, single base pair polymorphisms, transitions, transversions, inversions, copy number variations, duplications/deletions of long stretches of nucleic acids, and combinations of these.

### BRIEF DESCRIPTION OF THE DRAWING

Table 1 describes outcome data for lung cancer patients and detection of HER2 expression in tumor cells obtained from patient tumor tissue that also harbor a mutation in the HER2 gene. While all patient tumors harbored HER2 mutations, 6/8 patients whose tumors show expression of unique fragment peptides from the HER2 protein at any quantitative level demonstrated a therapeutic response to treatment with T-DM1. Two patients whose tumors harbor a HER2 mutation but where unique fragment peptides from the HER2 protein were not detected showed no response to treatment with T-DM1.

### DETAILED DESCRIPTION

Methods are provided for treating a patient with the HER2-targeted therapeutic agent trastuzumab emtansine (T-DM1) (or equivalent drug, as discussed above). Tumor tissue from a cancer patient, for example a lung cancer patient, is analyzed to determine whether the patient will clinically respond in a favorable manner to T-DM1 Specifically, a patient is treated with T-DM1 when analysis of tumor cells from the patient detects expression of HER2 protein and the presence of at least one mutation in the HER2 gene. Unlike the situation with conventional T-DM1 treatment, (see Wolff et al., (Arch Pathol Lab Med. 138:241-256 (2014) the HER2 gene does not need to be amplified, and HER2 does not need to be overexpressed for T-DM1 treatment to be effective, provided that a HER2 mutation is present. In fact, provided that HER2 expression is detectable at the limit of detection, the method is effective. Moreover, detection of Her3 expression in the tumor tissue can be used as a further indicator that treatment with T-DM1 will be effective. As with HER2, the level of Her3 expression need only be at or above the limit of detection for treatment with T-DM 1 to be effective.

The sample advantageously is formalin-fixed. Using a mass spectrometer, one or more unique specific peptide fragments are detected and quantified, and particular characteristics about the peptides from the HER2 protein in cells derived from formalin fixed paraffin embedded (FFPE) tissue is determined. Unique HER2-specific peptide fragments derive from the full-length HER2 protein. Surprisingly it has been found that HER2-specific peptides can be reliably detected and quantitated simultaneously in digests prepared from FFPE samples of tumor tissue. See U.S. Pat. No. 9,765,380, the contents of which are hereby incorporated by reference in their entirety. Detection of Her3 expression in the same sample can also be achieved using similar methods, as described, for example, in U.S. Pat. No. 9,128,102, the contents of which are hereby incorporated by reference in their entirety. The presence of mutations in the HER2 gene can be determined by methods that are known in the art, as further described below.

Detecting and measuring unique HER2-specific peptides and, optionally, HER3-specific peptides directly in complex protein lysate samples prepared from cells procured from patient tissue samples, such as formalin fixed cancer patient tissue, is performed using mass spectrometry. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue® reagents and protocol available from Expression Pathology Inc. (Rockville, Md.).

The most widely and advantageously available form of tissue, and cancer tissue, from cancer patients is formalin fixed, paraffin embedded tissue. Formaldehyde/formalin fixation of surgically removed tissue is by far and away the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

Results from a mass spectrometry analysis, and especially using the method of SRM/MRM mass spectrometry, can be used to correlate accurate and precise quantitative levels of the HER2 protein and, optionally, HER3 protein, within the specific cancer of the patient from whom the tissue was collected and preserved, including lung cancer tissue. This not only provides diagnostic/prognostic information about the cancer, but also permits a physician or other medical professional to determine appropriate therapy for the patient. In this case, utilizing a mass spectrometry assay for HER2 protein and, optionally, Her3 protein, can provide information about specific levels of HER2 (and HERS) protein expression in cancer tissue and be used as part of the methods described herein to determine whether or not the patient from whom the cancer tissue was obtained will respond in a favorable way to the therapeutic agent T-DM1.

Treating breast cancer patients with the HER2-targeted therapeutic agent T-DM1 is very effective for preventing cancer from growing and thus prolonging the lives of cancer patients. The HER2 protein is a membrane-bound protein that functions to receive pro-growth signals from outside the cell and send those pro-growth signals to the inside of the tumor cell which stimulates tumor cells to grow and divide. The therapeutic agent T-DM1 is a HER2-specific antibody-drug conjugate (trastuzumab+emtansine) that, when it binds to the extracellular domain region of the HER2 protein, provides three functions to inhibit tumor cell growth and ultimately to kill tumor cells. The first function is to bind to the HER2 extracellular domain, which inhibits the binding of growth proteins that impart pro-growth signals to the HER2 protein, thereby inhibiting its signal conversion function that normally sends the received signal inward. The second is to elicit an immunological response to the tumor cells. Because T-DM1 is an antibody it marks tumor cells as foreign and thus can initiate and/or maintain an immunological response. The third function is to deliver the toxic drug emtansine that is conjugated to the antibody region of T-DM1 to the tumor cell wherein the HER2 protein that is bound by T-DM1 can internalize and deliver emtansine to the inside of the cell thus causing tumor cell death.

The HER2 protein must be expressed in tumor cells for T-DM1 to have any effect and thus it is necessary to detect and/or quantitate HER2 expression in tumor cells from the putative patient. Presently, the most widely-used and applied methodology to determine protein presence in cancer patient tissue, especially FFPE tissue, is immunohistochemistry (IHC). IHC methodology utilizes an antibody to detect the protein of interest. The results of an IHC test are most often interpreted by a pathologist or histotechnologist. This interpretation is subjective and does not provide quantitative data that may be predictive of sensitivity to the therapeutic agent T-DM1 that targets HER2.

Research from the HER2 IHC test suggests results obtained from this and other such tests may be wrong or misleading. This is probably because different laboratories use different rules for classifying positive and negative IHC status. Each pathologist running the tests also may use different criteria to decide whether the results are positive or negative. In most cases, this happens when the test results are borderline, meaning that the results are neither strongly positive nor strongly negative. In other cases, tissue from one area of cancer tissue can test positive while tissue from a different area of the cancer tests negative. Inaccurate IHC test results may mean that patients diagnosed with cancer do not receive the best possible care. If all or part of a cancer is positive for a specific target oncoprotein but test results classify it as negative, physicians are unlikely to recommend the correct therapeutic treatment, even though the patient could potentially benefit from those agents. If a cancer is oncoprotein target negative but test results classify it as positive, physicians may recommend a specific therapeutic treatment, even though the patient is unlikely to get any benefits and is exposed to the agent's secondary risks.

Thus, there is great clinical value in the ability to correctly detect and evaluate quantitative levels of the HER2 protein in tumors, for example lung tumors, so that the patient will have the greatest chance of receiving the most optimal treatment.

Detection of peptides and determining quantitative levels of specified HER2 fragment peptides is performed in a mass spectrometer, and can be performed using the SRM/MRM methodology, whereby the SRM/MRM signature chromatographic peak area of each peptide is determined within a complex peptide mixture present in a Liquid Tissue® lysate (see U.S. Pat. No. 7,473,532, as described above). Quantitative levels of the HER2 protein, and other proteins, including HERS as discussed above, are then determined by the SRM/MRM methodology whereby the SRM/MRM signature chromatographic peak area of an individual specified peptide from the HER2 protein in one biological sample is compared to the SRM/MRM signature chromatographic peak area of a known amount of a “spiked” internal standard for each of the individual specified HER2 fragment peptides. In one embodiment, the internal standard is a synthetic version of the same exact HER2 fragment peptide where the synthetic peptide contains one or more amino acid residues labeled with one or more heavy isotopes. Such isotope labeled internal standards are synthesized so that mass spectrometry analysis generates a predictable and consistent SRM/MRM signature chromatographic peak that is different and distinct from the native HER2 fragment peptide chromatographic signature peak and which can be used as a comparator peak. Thus when the internal standard is spiked in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for fragment peptides are displayed according to the amount of protein analyzed per sample.

In order to develop the SRM/MRM assay for HER2 (and HER3 fragment peptides) additional information beyond simply the peptide sequence may be utilized by the mass spectrometer. That additional information is used to direct and instruct the mass spectrometer, (e.g., a triple quadrupole mass spectrometer) to perform the correct and focused analysis of the specified fragment peptide. A triple quadrupole mass spectrometer is presently the most suitable instrument for analyzing a single isolated target peptide within a very complex protein lysate that may consist of hundreds of thousands to millions of individual peptides from all the proteins contained within a cell. The additional information provides the triple quadrupole mass spectrometer with the correct directives to allow analysis of a single isolated target peptide within a very complex protein lysate that may consist of hundreds of thousands to millions of individual peptides from all the proteins contained within a cell. Although SRM/MRM assays can be developed and performed on any type of mass spectrometer, including a MALDI, ion trap, ion trap/quadrupole hybrid, or triple quadrupole, presently the most advantageous instrument platform for SRM/MRM assay is often considered to be a triple quadrupole instrument platform.

Detecting expression of the HER2 protein in patient tumor tissue can also be performed by mass spectrometry that does not use the SRM/MRM methodology. Other mass spectrometry instrumentation other than a triple quadrupole is used to perform a “global” profile by identifying the presence of as many peptides as possible in a single biological sample, and in this case a protein lysate prepared from formalin fixed patient tumor tissue. One advantageous mass spectrometry instrument (LC-MS/MS) for this purpose is an ion trap or ion trap/quadrupole hybrid.

To detect HER2 expression and also to determine an appropriate reference level for HER2 quantitation, tumor samples are obtained from a cohort of patients suffering from cancer, in this case lung cancer. The lung cancer tumor samples are formalin-fixed using standard methods and the level of HER2 in the samples is measured using the methods as described above. The tissue samples may also be examined using IHC and FISH using methods that are well known in the art. The patients in the cohort are determined to have at least one mutation in the HER2 gene and have been treated with T-DM1. The response of the patients is measured using methods that are well known in the art, for example by recording the overall survival of the patients at time intervals after treatment. A suitable reference level can be determined using statistical methods that are well known in the art, for example by determining the lowest p value of a log rank test. Once a reference level has been determined it can be used to identify those patients whose HER2 protein expression levels indicate that they may likely benefit from treatment with T-DM1. Levels of the HER2 protein in patient tumor samples typically are expressed in amol/μg, although other units can be used. The skilled artisan will recognize that a reference level can be expressed as a range around a central value, for example, +/−250, 150, 100, 50 or 25 amol/μg.

Surprisingly it has been found that expression of HER2 at any detectable level in tumor tissue is predictive of response to T-DM1 when a mutation is present in the HER2 gene. Accordingly, the HER2 protein need not be overexpressed and the HER2 gene need not be amplified for T-DM1 therapy to be effective. In addition, it has been found that simultaneous detection of Her3 expression is a further indicator (but not an independent indicator) of response to T-DM1. As with HER2, the Her3 expression need only be above the limit of detection.

In conventional treatment methods with trastuzumab or T-DM1, tumor tissue from a patient typically is evaluated for HER2 expression to determine eligibility for treatment. Clinical guidelines for these measurements are well known in the art, for example the ASCO guidelines (see, for example, Wolff et al., “Recommendations for human epidermal growth factor receptor 2 testing in breast cancer: American Society of Clinical Oncology/College of American Pathologists clinical practice guideline update. J. Clin. Oncol. 31: 3997-4013 (2013) and Arch Pathol Lab Med. 138:241-256 (2014). Methods for determining HER2 expression typically involve IHC, as discussed above. In these conventional treatment methods a patient is treated with T-DM1 only when the HER2 protein is found to be overexpressed. In the context of the methods described herein, HER2 is considered to not be overexpressed when the level of HER2 expression is below the level of expression recommended for treatment under the ASCO guidelines, or where the patient would not otherwise qualify for treatment under these guidelines.

The detection of DNA mutations in the HER2 gene present in patient tumor cells from patient tumor tissue may be performed by detecting changes and/or variants from normal in the nucleic acid sequence from patient tumor cells collected using next generation sequencing (NGS) technology, whereby NGS technology is utilized to sequence an entire genome (whole genome sequencing [WGS]), sequence the entire collection of all the exons from all the genes present in a genome (whole exome sequencing [WES]), or sequence a pre-defined subset of the entire collection of exons from all the genes present in a genome (exome sequencing [ES]). Methods for sequencing the HER2 gene and identifying the presence of mutations are known in the art.

Because both nucleic acids and protein can be analyzed from the same Liquid Tissue® biomolecular preparation it is possible to generate additional information about disease diagnosis and drug treatment decisions from the nucleic acids in same sample upon which proteins were analyzed. For example, if the HER2 protein is expressed by certain cells at increased levels, when assayed by SRM the data can provide information about the state of the cells and their potential for uncontrolled growth. At the same time, information about the mutational status of genes can be obtained from nucleic acids present in the same Liquid Tissue® biomolecular preparation. Nucleic acids can be assessed simultaneously to the SRM analysis of proteins, including the HER2 protein. In one embodiment, information about HER2 protein expression can be combined with information about the sequence of the HER2 gene and whether or not there is at least one mutation in the HER2 gene. In addition, nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

1. A method of treating a patient suffering from cancer, comprising:
a. detecting and quantifying a level of the HER2 protein in a tumor sample obtained from the patient, wherein said tumor sample comprises one or more mutations in the HER2 gene;
b. treating the patient with a first therapeutic regimen comprising an effective amount of therapeutic agent trastuzumab emtansine (T-DM1) when HER2 protein is detected, and wherein said HER2 protein is not overexpressed under ASCO guidelines or
c. treating the patient with a second therapeutic regimen that does not comprise an effective amount of T-DM1 when HER2 is not detected.

2. The method of claim 1, further comprising detecting expression of HER3 protein, wherein the patient is treated with said first therapeutic regimen comprising an effective amount of the therapeutic agent trastuzumab emtansine (T-DM1) when HER2 expression is detected, and wherein HER2 is not overexpressed under the ASCO guidelines and when HER3 is detected.

3. The method of claim 1, wherein the HER2 protein is detected and quantified by detecting and quantifying a HER2 fragment peptide by mass spectrometry in a protein digest of the tumor sample.

4. The method of claim 2, wherein the HER3 protein is detected by detecting a HER3 fragment peptide by mass spectrometry in a protein digest of the tumor sample.

5. The method of claim 3, wherein said protein digest comprises a protease digest.

6. The method of claim 5, wherein said protein digest comprises a trypsin digest.

7. The method of claim 3, wherein mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

8. The method of claim 7, wherein a mode of mass spectrometry is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

9. The method of claim 1, wherein the tumor sample is solid tissue.

10. The method of claim 9, wherein the solid tissue is formalin fixed solid tissue.

11. The method of claim 10, wherein the formalin fixed solid tissue is paraffin embedded tissue.

12. The method of claim 3, wherein quantifying the HER2 fragment peptide comprises determining the amount of the HER2 fragment peptide in the tumor sample by comparing to an internal standard peptide of known amount.

13. The method of claim 12, wherein the internal standard peptide is an isotopically labeled peptide.

14. The method of claim 13, wherein the isotopically labeled peptide comprises one or more heavy stable isotopes selected from ¹⁸O, ¹⁷O, ¹⁵N, ¹³C, ²H and a combination thereof.

15. The method of claim 3, wherein detecting and quantifying the HER 2 fragment peptide can be combined with detecting and quantifying other peptides from other proteins in multiplex.

16. The method of claim 1, wherein the one more mutations are detected within the HER2 gene using one or more methods selected from the group consisting of standard nucleic acid sequencing, next generation nucleic acid sequencing, polymerase chain reaction, restriction fragment polymorphism analysis, fluorescent in-situ hybridization (FISH), and a combination thereof.

17. The method of claim 1, wherein said one or more mutations in the HER2 gene is selected from the group consisting of single nucleotide changes, insertions, deletions, rearrangements, duplications, duplications/deletions of individual nucleotides, duplications/deletions of multiple nucleotides, single base pair polymorphisms, transitions, transversions, inversions, copy number variations, duplications/deletions of long stretches of nucleic acids, and a combinations thereof.

18. The method of claim 4, wherein said protein digest comprises a protease digest.

19. The method of claim 18, wherein said protein digest comprises a trypsin digest.

20. The method of claim 4, wherein mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

21. The method of claim 20, wherein a mode of mass spectrometry is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).
